(19) United States
(12) Patent Application Publication
(10) Pub. No.: US 2009/0287264 A1
(43) Pub. Date: Nov. 19, 2009

(54) APPLIANCE AND SEQUENCE OF PULSES FOR THE APPLICATION OF ELECTROTHERAPY

(76) Inventor: Robert Paret, Oostende (BE)

Correspondence Address: BROWDY AND NEIMARK, P.L.L.C.;624 NINTH STREET, NW, SUITE 300, WASHINGTON, DC, 20001-5303, US

(21) Appl. No.: 12/296,281

(22) PCT Filed: Apr. 5, 2007
(86) PCT No.: PCT/IB2007/051226

§ 371 Date: Jun. 1, 2009

(30) Foreign Application Priority Data
Apr. 5, 2006 (BE) 2006/0213

(51) Int. Cl. A61N 1/36; A61F 7/00

(52) U.S. Cl. 607/3; 607/48; 607/72

(57) ABSTRACT

The invention relates to a transcutaneous nerve and/or muscle stimulation appliance with electrodes destined to apply a sequence of electric pulses to the skin of a person, providing means that allow the generation of multiphase pulses during a stimulation period, these means allowing the generation of successive sequences of the above-mentioned multiphase pulses during successive stimulation periods with a frequency between 10 Hz and 100 Hz, more in particular a frequency of approximately 50 Hz, these means furthermore allowing a rest period between successive stimulation periods, during which rest period a sequence of electric pulses is generated with a frequency ranging from 0.5 Hz to 5 Hz and with an amplitude lower than that of the above-mentioned multiphase pulses.

The invention relates to a transcutaneous muscle and/or nerve stimulation appliance with means destined to apply a sequence of electric pulses to the skin of a person by means of electrodes. More in particular, these means allow the generation of multiphase pulses during a stimulation period, with a first phase that has a certain voltage during 5 to 20 milliseconds, preferably during 10 milliseconds, and a second phase that has a certain voltage during 5 to 20 milliseconds, preferably during 10 milliseconds.

The use of such a state-of-the-art appliance has a number of disadvantages for the user. For example, it has become clear that, when a sequence of electric pulses are applied to stimulate a person's muscles to train them, for instance, muscular stiffness occurs after a relatively short period of time. It is assumed that one of the causes of this stiffness is an accumulation of lactic acid in the muscles, leading to muscle acidification.

The invention wants to remedy these disadvantages by submitting an appliance for the electric stimulation of the nerves and/or muscles of a person, almost completely avoiding the occurrence of muscle stiffness. This appliance therefore enables a much more efficient and intensive treatment of a person.

For this purpose the above-mentioned means allow the generation of successive sequences of the above-mentioned multiphase pulses during successive stimulation periods, with a frequency between 10 Hz and 100 Hz, more in particular a frequency of approximately 50 Hz, these means furthermore allowing a rest period between successive stimulation periods, during which rest period a sequence of electric pulses is generated with a frequency ranging from 0.5 Hz to 5 Hz and with an amplitude lower than that of the above-mentioned multiphase pulses.

The above-mentioned means allow the generation of two-phase rest pulses, with a first phase and a second phase of 5 to 20 milliseconds each, but preferably with a duration of approximately 10 milliseconds.

The above-mentioned rest period preferably has a duration between 4 and 15 seconds, during which the above-mentioned rest pulses are generated.

In the preferred version of the appliance, on the basis of the invention, the amplitude of the above-mentioned generated rest pulses is smaller than one fourth of the amplitude of the above-mentioned multiphase pulses applied during the stimulation period.

The above-mentioned means preferably allow the generation of multiphase pulses, which are applied during the stimulation period and of which the maximum voltage does not exceed 250 volts and ranges in particular between 10 and 50 volts, preferably between 20 and 30 volts.

In an interesting version of the appliance, on the basis of the invention, this generates pulses that have an almost rectangular appearance.

Other particulars and advantages of the invention will appear from the description given below of a few specific versions of the appliance on the basis of the invention; this description is only given by way of example and does not limit the scope of the required protection; the digits used in the description refer to the figures attached to this document.

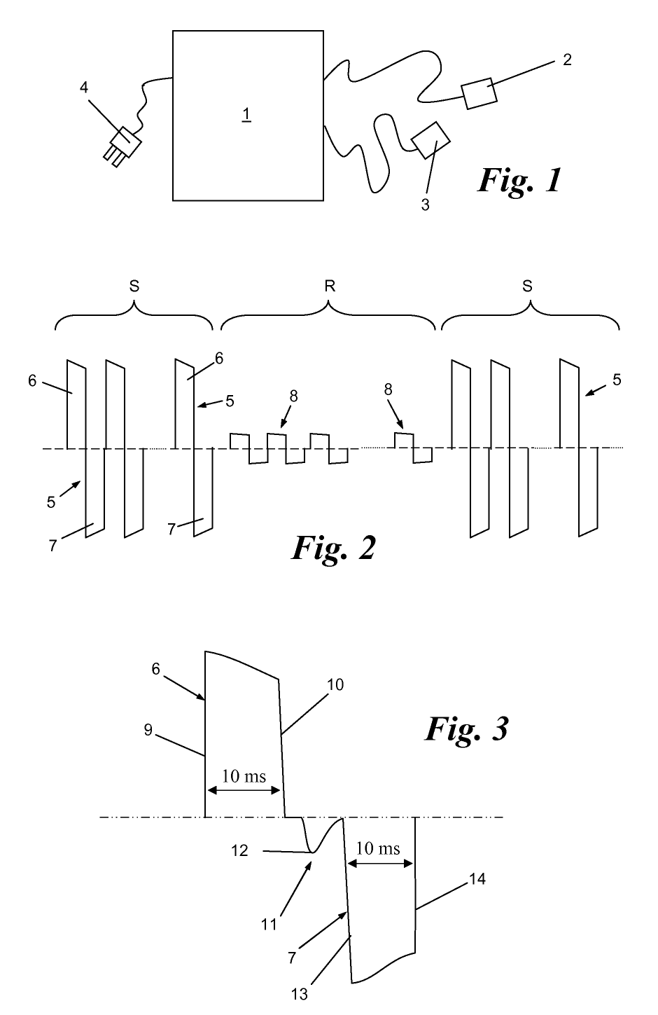

FIG. 1 is a schematic representation of a nerve stimulation appliance, on the basis of the invention, with two electrodes.

FIG. 2 is a schematic representation of a pulse sequence before and during a stimulation period, a rest period and another stimulation period.

FIG. 3 is a schematic representation of a pulse generated by the nerve stimulation appliance, on the basis of the invention.

In the figures the same digits refer to the same or analogous elements.

The nerve stimulation appliance 1, on the basis of the invention, as represented in FIG. 1, is equipped with two electrodes 2 and 3 and is connected to the mains with plug 4. These electrodes 2 and 3 must be placed on the skin of a person to be treated so that electric pulses can be applied by means of the nerve stimulation appliance.

As described below, this appliance allows the generation of rectangular multiphase pulses during a stimulation period, a first phase with a positive voltage for 5 to 20 milliseconds and a second phase with a negative voltage for 5 to 20 milliseconds.

It is found that by applying such electric pulses to the skin of a person a contraction, and therefore stimulation, of practically all muscle fibres is achieved. In this manner it is possible to train the muscles very intensively by means of the appliance on the basis of the invention. However, this causes an accumulation of lactic acid in the muscles, leading to muscle acidification and therefore muscular pain.

The problem of muscle acidification is reduced when pulses are applied during a period that is considerably shorter than 5 milliseconds. In such a case it is assumed that part of the muscle fibres of a muscle does not contract and is therefore not stimulated. This means that the muscles are stimulated less intensively so that the quantity of lactic acid accumulated is smaller than in the above-mentioned very intensive training and can more easily be removed.

When the appliance based on the invention is used for such an intensive training, it is required to introduce a rest period at regular intervals in order to remove the accumulated lactic acid from the muscles. Surprisingly however, it was found that if, during this rest period, an electric pulse with an amplitude lower than that used during the stimulation period is applied to the muscles, the lactic acid is removed more quickly.

Thus the appliance based on the invention contains means allowing the generation of successive sequences of the above-mentioned multiphase pulses during successive stimulation periods, with a frequency between 10 Hz and 100 Hz. This frequency may for instance be approximately 50 Hz. These means furthermore allow a rest period between successive stimulation periods, during which a sequence of electric rest pulses with a frequency between 0.5 Hz and 5 Hz is generated. The amplitude of these rest pulses is considerably lower than that of the above-mentioned multiphase pulses of the stimulation period and may be for instance less than one fourth of these pulses of the above-mentioned stimulation period.

FIG. 2 is a schematic representation, given by way of example, of a sequence of electric pulses generated by the appliance based on the invention. This figure represents, from left to right, a stimulation period S, a rest period R and another stimulation period S.

During each of the stimulation periods S, for instance, a sequence of multiphase pulses 5 is generated, with a first phase 6 during 10 ms and a maximum voltage of 10 to 50 volts. A second phase 7 of these pulses 5 also has a duration of 10 ms and an opposite voltage of 10 to 50 volts maximally. The sequence of multiphase pulses 5 of the stimulation period is obtained by repeating this multiphase pulse 5 with a frequency ranging from 10 to 100 Hz. The multiphase pulses 5 is followed by a rest period R during which so-called electric rest pulses 8 are generated. These rest pulses may have the same duration as the pulses 5 generated during the stimulation period S. The duration of the rest pulses 8 can for instance be 10 ms.

However, the intensity and the frequency of the rest pulses 8 should be lower than those of the above-mentioned multiphase pulses 5 applied during the stimulation period S. During the rest period, pulses 8 with a frequency of preferably 1 to 2 Hz for muscle stimulation are generated.

Furthermore, the pulses 8 generated during the rest period R may have the same shape as the pulses 5 of the stimulation period S.

The purpose of this muscle stimulation during the rest period R is to cause clonic muscle contractions with an intensity that is as low as possible in order to influence the venous and lymphatic system through the muscle pump, so that lactates are removed and muscle acidification is prevented.

The duration of the rest period R applied by the appliance based on the invention is preferably between 4 and 15 seconds. The duration that is chosen may depend on the type of muscle fibres one wishes to train. In most cases, a rest period R of 6 to 12 seconds is opted for.

FIG. 3 is a schematic representation of a multiphase pulse 5 with a rectangular shape, which appears to be highly efficient for the rehabilitation of paralysed or weakened muscles, pain alleviation or the acceleration of a healing process, for instance, and which is applied by the appliance based on the invention during the above-mentioned stimulation period. This pulse 5 also appears to be highly suitable for intensive muscle training.

This pulse consists of three phases. The first phase 6 has a rectangular shape and a steep, practically vertical, flank 9, with an increase in voltage from 0 volt to almost 7 volts. During a period of approximately 10 milliseconds the voltage is maintained between 6 and 7 volts. After this period of 10 milliseconds the voltage drops to 0 volt again, via a practically vertical flank 10.

Subsequently, this pulse 5 has a compensating phase 11 with a small negative peak of approximately 1.2 volts during 5 milliseconds. The compensating phase 11 may consist of a so-called decay of the first phase 6.

Finally, the pulse has a next phase 7, which is also practically rectangular in shape with a steep, almost vertical downward flank 13 and with a drop of the voltage from 0 volt to almost −7 volts. During a period of approximately 10 milliseconds the voltage is maintained between −6 and −7 volts. After this period of 10 milliseconds the voltage increased to 0 volt via a practically vertical flank 14.

Good results were obtained when a pulse was used with a compensating phase 11 with a voltage peak of less than one third of the voltage of the preceding phase 6 and the following phase 7. The duration of this compensating phase 11 is between 2 and 10 milliseconds.

The voltage of phases 6 an 7 may vary between 0 and 250 volts, preferably between 10 and 30 volts. The best results are obtained with a voltage between 20 and 50 volts.

The duration of these phases 6 and 7 may vary between 5 milliseconds and 20 milliseconds and is preferably between 5 and 15 milliseconds.

The nerves and muscles of the person to be treated are stimulated by means of a sequence of such pulses, which are applied through the above-mentioned electrodes 2 and 3, which are positioned on the skin of the person concerned.

The frequency of the sequence of these pulses 5 is adjustable and is preferably between 10 Hz and 100 Hz. In order to avoid habituation to the pulses, resulting in a decreased healing or pain-alleviating effect, this frequency is modulated advantageously so that the interval between two successive pulses varies.

An appliance allowing the generation of a multiphase pulse 5 as represented in FIG. 3 is described in patent application WO 02/18008.

Another version of the appliance, based on the invention, allows the generation of a rectangular two-phase symmetric pulse 5 which does not include the above-mentioned compensating phase 11. This pulse 5 therefore consists of a first rectangular phase 6 immediately followed by a second rectangular phase 7, as represented in FIG. 2.

It was furthermore found that when the skin of the person to be treated is warm, electric pulses penetrate deeper into the skin and the muscle tissue. By warming the surface of the skin to be treated and applying electric pulses at the same time, the irritability of the muscles is decreased and microtraumas are avoided. In addition, waste products of the muscles are removed better and no or only a slight degree of muscle acidification occurs.

Therefore, the above-mentioned electrodes 2 and 3 are advantageously equipped with a heating element.

Obviously, the invention is not limited to the versions of the appliance and the electrodes described above and represented in the figures. For instance, more than two electrodes can be connected to the outputs of the appliance based on the invention. It is also possible to connect the nerve stimulation appliance to multifunctional, either or not touch sensitive screens or light emitting diodes in order to read certain variables such as the duration of the electric frequency-controlled pulses, the size of the pulses and the temperature of the electrodes, whereby the value of these variables can be adjusted to the person to be treated.

1. Transcutaneous nerve and/or muscle stimulation appliance with electrodes destined to apply a sequence of electric pulses to the skin of a person, providing means that allow the generation of multiphase pulses during a stimulation period, with a first phase that has a certain voltage during 5 to 20 milliseconds, preferably during 10 milliseconds, and a second phase that has a certain voltage during 5 to 20 milliseconds, preferably during 10 milliseconds, characterised by the fact that the above-mentioned means allow the generation of successive sequences of the above-mentioned multiphase pulses during successive stimulation periods, with a frequency between 10 Hz and 100 Hz, more in particular a frequency of approximately 50 Hz, these means furthermore allowing a rest period between successive stimulation periods, during which rest period a sequence of electric rest pulses is generated with a frequency ranging from 0.5 Hz to 5 Hz and with an amplitude lower than that of the above-mentioned multiphase pulses.

2. Appliance according to claim 1, characterised by the fact that the above-mentioned means allow the generation of two-phase rest pulses with a first phase and second phase with a duration of 5 to 20 milliseconds each, preferably a duration of approximately 10 milliseconds.

3. Appliance according to claim 1, characterised by the fact that the above-mentioned rest period has a duration between 4 and 15 seconds, during which the above-mentioned rest pulses are generated.

4. Appliance according to claim 1, characterised by the fact that the above-mentioned rest period has a duration of 6 to 12 seconds.

5. Appliance according to claim 1, characterised by the fact that the above-mentioned stimulation period has a duration of 10 to 100 seconds.

6. Appliance according to claim 1, characterised by the fact that the amplitude of the above-mentioned rest pulses does not exceed one fourth of that of the above-mentioned multiphase pulses applied during the stimulation period.

7. Appliance according to claim 1, characterised by the fact that the above-mentioned means allow the generation of multiphase pulses applied during the stimulation period, the maximum voltage of which is lower than 250 volts and more in particular varies between 10 and 50 volts, preferably between 20 and 30 volts.

8. Appliance according to claim 1, characterised by the fact that the above-mentioned multiphase pulses have an almost rectangular shape.

9. Appliance according to claim 1, characterised by the fact that the voltage of the above-mentioned first phase of the pulses generated during the stimulation period or during the above-mentioned rest period is opposite to the voltage of the second phase of these pulses.

10. Appliance according to claim 1, characterised by the fact that the above-mentioned means are executed in such a manner that during the stimulation period, between the above-mentioned first phase and the above-mentioned second phase, a compensating phase is generated with a voltage lower than one third of the voltage of the first and/or second phase and with a duration between 2 and 10 milliseconds.

11. Appliance according to claim 1, characterised by the fact that it contains a heating element that allows the warming of the skin and/or the underlying muscle tissue at a temperature between 37° C. and 55° C. while the above-mentioned pulses are applied.

12. Sequence of electric pulses, with multiphase pulses during a stimulation period with a frequency between 10 Hz and 100 Hz, in particular a frequency of approximately 50 Hz, these multiphase pulses consisting of a first phase with a certain voltage between 5 and 20 milliseconds, preferably during 10 milliseconds, and a second phase with a certain voltage between 5 and 20 milliseconds, preferably during 10 milliseconds, this pulse sequence consisting, during a rest period immediately following the above-mentioned stimulation period, of electric rest pulses with a frequency varying between 0.5 Hz and 5 Hz and with an amplitude lower than that of the above-mentioned multiphase pulses.

13. Sequence of electric pulses, in particular according to conclusion 12, for the transcutaneous stimulation of nerves or muscles of a person, these pulses being multiphase pulses, consisting of a first phase with a positive voltage during 5 to 20 milliseconds, in particular during 5 to 15 milliseconds and a second phase with a negative voltage during 5 to 20 milliseconds, in particular during 5 to 15 milliseconds.

14. Sequence of electric pulses according to conclusion 13, characterised by the fact that between the above-mentioned first phase and the above-mentioned second phase of the pulses a compensating phase is applied with a voltage lower than one third of the voltage of the first and/or second phase.

15. Sequence of electric pulses according to conclusion 13, characterised by the fact that the above-mentioned compensating phase has a duration between 2 and 10 ms.
